(19) United States
(12) Patent Application Publication
(10) Pub. No.: US 2022/0193372 A1
(43) Pub. Date: Jun. 23, 2022

(54) DUAL ARTICULATING CATHETER

(71) Applicant: Covidien LP, Mansfield, MA (US)

(72) Inventors: John W. Komp, Dillon, CO (US); Nathan J. Knutson, Long Lake, MN (US); Jane L. Kohen, Minneapolis, MN (US)

(21) Appl. No.: 17/544,910

(22) Filed: Dec. 7, 2021

Related U.S. Application Data
(60) Provisional application No. 63/128,897, filed on Dec. 22, 2020.

(51) Int. Cl. A61M 25/01

(52) U.S. Cl. CPC A61M 25/0147; A61M 2025/0166

(57) ABSTRACT

An articulating catheter defining a first curve at a distal portion of the catheter and a second curve proximal of the first curve.

### BACKGROUND

### Technical Field

This disclosure relates to the field of navigation catheters and particularly to navigation catheters capable of achieving a dual articulation and a user defined curvature to promote navigation and target acquisition.

### Description of Related Art

There are several commonly applied medical methods, such as endoscopic procedures or minimally invasive procedures, for treating various maladies affecting organs including the liver, brain, heart, lungs, gall bladder, kidneys, and bones. Often, one or more imaging modalities, such as magnetic resonance imaging (MRI), ultrasound imaging, computed tomography (CT), or fluoroscopy are employed by clinicians to identify and navigate to areas of interest within a patient and ultimately a target for biopsy or treatment. In some procedures, pre-operative scans may be utilized for target identification and intraoperative guidance. However, real-time imaging may be required to obtain a more accurate and current image of the target area. Furthermore, real-time image data displaying the current location of a medical device with respect to the target and its surroundings may be needed to navigate the medical device to the target in a safe and accurate manner (e.g., without causing damage to other organs or tissue).

For example, an endoscopic approach has proven useful in navigating to areas of interest within a patient, and particularly so for areas within luminal networks of the body such as the lungs. To enable the endoscopic approach, and more particularly the bronchoscopic approach in the lungs, endobronchial navigation systems have been developed that use previously acquired MRI data or CT image data to generate a three-dimensional (3D) rendering, model, or volume of the particular body part such as the lungs.

The resulting volume generated from the MRI scan or CT scan may be utilized to create a navigation plan to facilitate the advancement of a navigation catheter (or other suitable medical device) through a bronchoscope and a branch of the bronchus of a patient to an area of interest. A locating or tracking system, such as an electromagnetic (EM) tracking system, may be utilized in conjunction with, for example, CT data, to facilitate guidance of the navigation catheter through the branch of the bronchus to the area of interest. In certain instances, the navigation catheter may be positioned within one of the airways of the branched luminal networks adjacent to, or within, the area of interest to provide access for one or more medical instruments.

Accurate placement of the catheter is important to ensure that tools such as biopsy and treatment tools interact with the desired tissue. Improvements to current navigation catheter systems are desired.

### SUMMARY

One aspect of the disclosure is directed to

### BRIEF DESCRIPTION OF THE DRAWINGS

Various aspects and embodiments of the disclosure are described hereinbelow with references to the drawings, wherein:

FIGS. 1A-1C depict the distal portion of catheters having a pre-shaped curvature;

FIGS. 2A-2C depict a distal portion of a catheter having a variable curvature in accordance with the disclosure;

FIG. 3 depicts an articulating mechanism for incorporation in a catheter in accordance with the disclosure;

FIG. 4 depicts a distal portion of a catheter having an alternative articulating mechanism in accordance with the disclosure;

FIG. 5 depicts a drive mechanism for acting one or more pull wires in accordance with the disclosure;

FIG. 6 depicts an alternative drive mechanism for acting one or more pull wires in accordance with the disclosure;

FIG. 7 depicts a further alternative for acting one or more pull wires in accordance with the disclosure;

FIG. 8 depicts an intraluminal navigation system in accordance with the disclosure;

FIG. 9 depicts a schematic view of a workstation for utilization with an intraluminal navigation system in accordance with the disclosure.

### DETAILED DESCRIPTION

The disclosure is directed to an articulating catheter for use in navigation of luminal networks such as the airways of the lungs. Articulation, particularly dual point articulation allows for a user to ensure orientation of the distal portion of the catheter in a desired direction. This orientation can be particularly useful when passing tools such as biopsy and therapy tools (e.g., microwave ablation catheters through the catheter to diagnose or treat desired tissue. The use of two articulation points allows for acute manipulation of the distal portion of the catheter as well as a gentler articulation of a more proximal portion of the catheter. In combination, the dual articulation mechanism allows for the catheter to assume a variety curvatures and shapes to improve positioning of the catheter. To reduce the complexity of the design a two-point articulation system can be achieved using just a single guidewire.

Catheters typically have either a fixed or adjustable curvature at the distal tip. The center of this curvature will usually be back 2-4 centimeters from the distal tip and have a curvature radius of 1-2 centimeters. This curvature is used to aid endoluminal navigation by pointing the distal tip towards a luminal branch. The user rotates the catheter in order to align the tip with the desired branch and then advances the catheter into the branch.

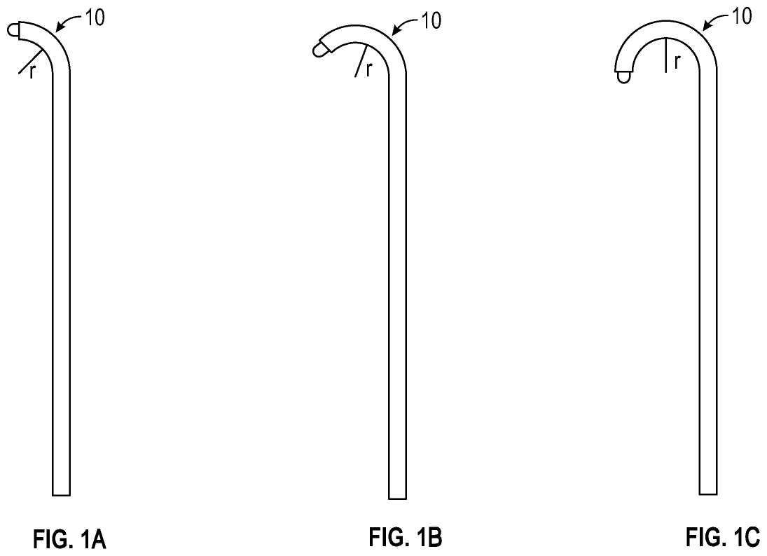

FIGS. 1A-1C depicts versions of a common curved catheter 10 used for navigation within the airways of a patient. The catheter 10 has in FIG. 1A a 45° curve, in FIG. 1B a 90° curve, and in FIG. 1C a 180° curve located at the distal portions of the catheter 10. Differing amounts of curvature in the catheter may be used to navigate to differing portions of a patient's airways. For example, a catheter including 180° curve may be used for navigating to a posterior portion of the upper lobe of the patient's airways. In practice, by a rotate and push process, a clinician can manipulate the curve in the catheter 10 such that it is oriented to a desired lumen in the airways. Once so oriented, pushing or advancing of the catheter ensures that the desired airway is entered. This process can be repeated many times to reach a desired location in the airways following a pathway plan as described in greater detail below.

While such broad curvature is useful for luminal navigation, it does not aid in pointing the distal tip off the general axis of the catheter. This is especially true in smaller lumens where the walls of the lumen resist the broad curvature. In such cases a much sharper means of articulating the distal tip is provides the needed direction change. This articulation is typically within the first 0.5-1 centimeter of the distal tip and has a sharp articulation of 5-30 degrees. This smaller articulation can also be useful in combination with the broader curvature during navigation.

There are two additional aspects of the pre-curved catheter 10 that can be challenging. First, the clinician must decide which catheter 10 to use through the procedure and once selected must utilize that catheter 10 through the entire procedure. Secondly, in the last few centimeters of navigation, the curvature which has to this point assisted in navigation makes it challenging to align the opening in the catheter 10 with the target. Rotation of the curved catheter 10 results in the distal end and the opening formed therein rotating in an arc around the target rather than changing alignment to coincide with the target.

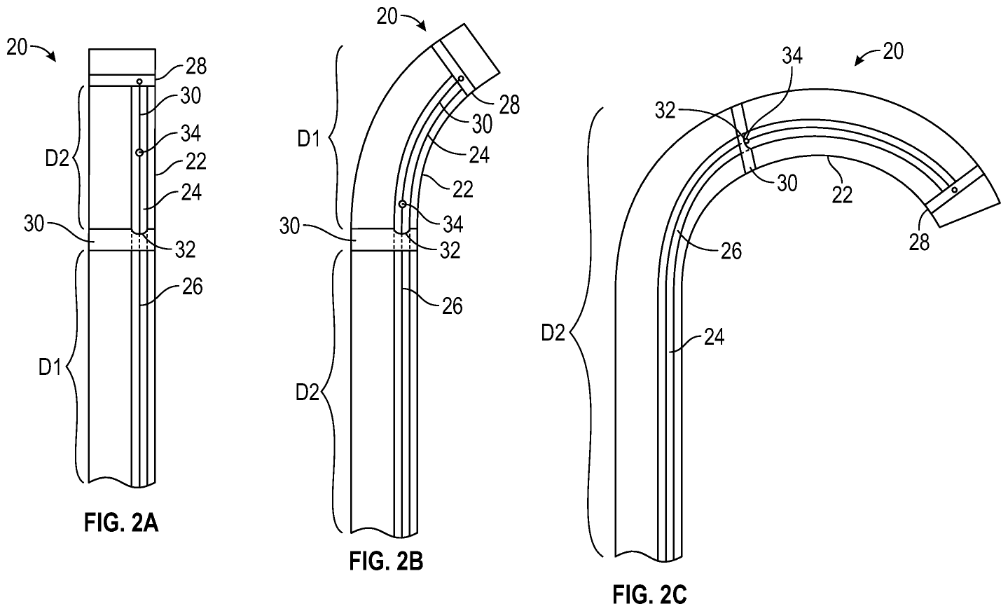

A catheter can be built that combines two types of articulation. A pull wire implementation using one or a pair of pull wires for the articulation joints can be employed. The pull wires articulate in substantially a single one plane. To achieve curvatures similar to those depicted in FIGS. 2A-2C in an articulating catheter and achieving improved alignment characteristics.

FIGS. 2A-2C depict a catheter 20 in accordance with the disclosure having two points of articulation. The catheter 20 includes a catheter tube 22. Formed in the catheter tube 22 is a pull wire lumen 24 allowing for passage of a pull wire 26. Also formed in the catheter tube 22 are a first pull ring 28 to which the pull wire 26 is permanently secured (e.g., by welding, gluing or other means) and a second pull ring 30. The second pull ring 30 includes an orifice 32 through which the pull wire 26 passes. Formed on the pull wire 26 is a stopper 34. As the pull wire 26 is pulled proximally the pull wire 26 acts on the first pull ring 28. The result of this initial translation of the pull wire 26 proximally (e.g., towards the user) is to change the shape of a the distal most portion D2 of the catheter 20 as can be seen in FIG. 2B. The pull wire 26 translates through the orifice 32 in the second pull ring 30 but does not change the shape of the catheter 20 at the second pull ring 30. Further retraction of the pull wire 26 causes the stopper 34 to reach the orifice 32, but because it is sized not to pass through the orifice 32. Tensioning the pull wire 26 once the stopper 34 is secured in the orifice 32 causes the catheter tube 22 to buckle and create a second curve at the second pull ring 30 as can be seen in FIG. 2C. The spacing between the position of the stopper 34 when the catheter 20 is straight (FIG. 2A) and the point at which the stopper 34 is received in the orifice 32 defines the delay between the delay between the start of articulation of the distal portion D2 and the start of the articulation of more proximal portion D1.

In one embodiment, the catheter 22 may be formed from materials having a uniform durometer rating along the entire length. However, the catheter tube 22 may have different stiffnesses in regions D1 and D2 to allow for different amounts of articulation of the catheter 20. In some embodiments, the proximal portion D1 may be formed from materials having a Shore A durometer ranging from about 30 to about 80. The distal portion D2 may be formed from materials having a Shore A durometer rating from about 30 to about 63. As will be appreciated other ranges may also be employed without departing from the scope of the disclosure. In a further embodiment, the stopper 34 may be eliminated and the two curves can be achieved relying on the difference in stiffness between the distal portion D2 and the proximal portion Dl.

The differences in material choices can result in the catheter 20 having a memory. That is, upon release of the tension on the pull wire 26, the catheter 20 has a tendency to return to its unarticulated shape (e.g., straight as depicted in FIG. 2A). Additionally or alternatively, the pull wire 26 may be formed of a material that has a tendency to return to its unstressed state (e.g., nitinol). Still further, the pull wire may be selected from a material that has sufficient column strength to allow the pull wire 26 to push the catheter 20 to return to its unarticulated position.

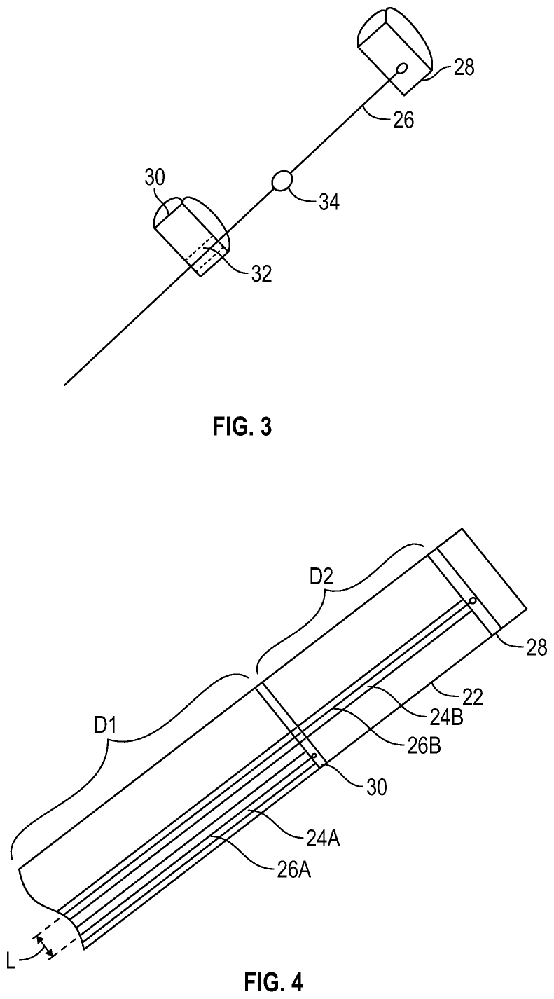

FIG. 3 depicts a perspective view of the actuation mechanism of the catheter 20 with the catheter tube 22 removed to more clearly depict the interaction of the components. The stopper 34 may be adhered to the pull wire 26 via an adhesive, swaged to the pull wire 26, knotted to the pull wire 26, or welded to the pull wire 26. Additionally or alternatively, the stopper 34 may have a profile that is vastly different from the profile of the orifice 32. For example, the stopper 34 may be rectangular or cross shaped while the orifice 32 is substantially round.

Those of skill in the art will recognize that an actuation mechanism may be deployed on a proximal end of the catheter 20. Some motorized actuation mechanisms are described in greater detail herein below in connection with a two-pull wire system, and those motorized actuation mechanisms may also be deployed in a single pull wire system of FIGS. 2A-2C. In addition, the actuation mechanism may be a manual actuation mechanism such as a lever system mounted on a proximal portion of the catheter 20 and configured to act on the pull wire 26 to retract the pull wire in a proximal direction to achieve the curvature of the catheter 20.

An alternatively embodiment of the disclosure can be seen in FIG. 4. While there are benefits to a single pull wire embodiment as depicted in FIGS. 2A-3, a two pull wire system can also be employed. As shown in FIG. 4 there is depicted a catheter 40 having a catheter tube 22 and two pull rings 28 and 30. The catheter 40 includes two pull wire lumens 24A and 24B. Running in the first pull wire lumen 24A is a pull wire 26A permanently affixed to the second pull ring 30. In the second pull wire lumen 24B is a second pull wire 26B which is permanently affixed to the first pull ring 28. In such a configuration either the first pull wire 26A or second pull wire 26B may be retracted or pull proximally to achieve a desired curvature or dual curvature of the catheter 40. The pull wires 26A and 26B should be separated by a distance L that is as small a practical so that the forces applied to the catheter tube 22 by the pull wire 26A and 26B are substantially aligned to achieve the desired curvature.

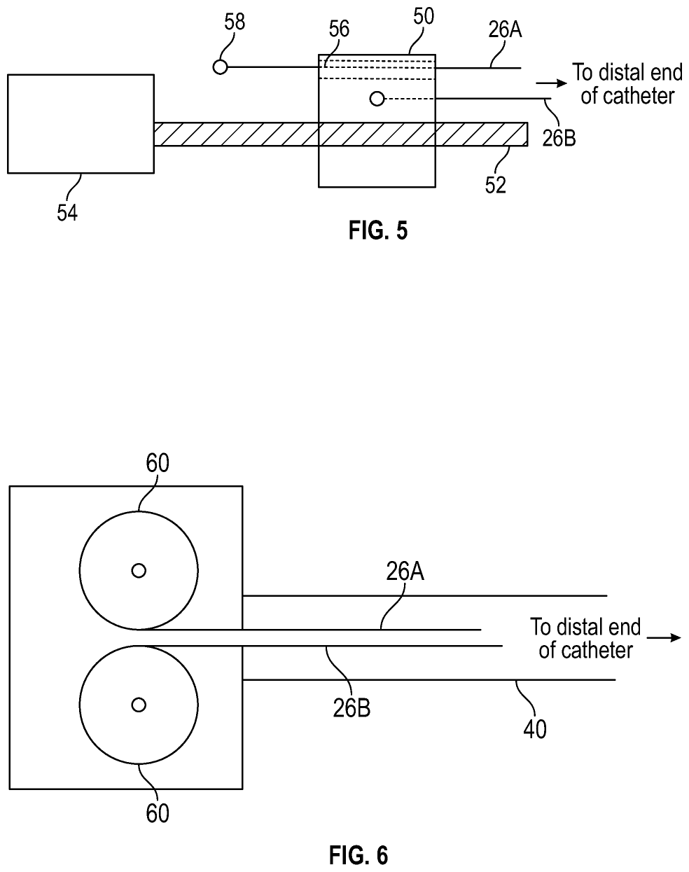

FIG. 5 depicts an exemplary actuation mechanism for the embodiment of FIG. 4 employing two pull wires 26A and 26B. As shown in FIG. 5, a shuttle 50 rides on a leadscrew 52. The leadscrew 52 may be driven by a motor 54. As the leadscrew 52 is rotated the shuttle 50, which has matching threads to the leadscrew 52, either advances in the direction of the distal end of the catheter or retracts towards the motor 54. As the shuttle 50 retracts in the direction of the motor 54 the pull wire 26B, which is permanently attached to pull ring 28 at the distal end of the catheter 40 and permanently attached to the shuttle 50 causes the distal portion of the catheter to curve to a position similar to what is shown in FIG. 2B. Pull wire 26A which is attached to pull ring 30 is slidingly engaged in a lumen 56 formed in the shuttle 50. Further retraction of the shuttle in the direction of the motor 54 causes a stopper 58 formed on the proximal end of the pull wire 26A to abut the lumen 56, but the stopper 58 is sized or shaped to prevent its passage through the lumen 56. Further retraction of the shuttle causes the catheter 40 to take on a shape similar to what is depicted in FIG. 2C. In this manner, the shuttle 50 acting on two pull wires 26A and 26B enables the catheter to flex in a similar manner as the single pull wire system shown in FIGS. 2A-3.

Those of ordinary skill in the art will recognize that the shuttle 50, leadscrew 52 and motor 54 may also be employed on a proximal end of catheter 20. But the shuttle 50 will have just a single pull wire 26 permanently affixed to the shuttle. Retraction of the shuttle 50 then causes the changes in shape from FIG. 2A-2C as the pull wire 26 is retracted acting first on the first pull ring 28 and then on the second pull ring 30 as the stopper 34 engages the orifice 32 as described above.

A further alternative embodiment using the two-pull wire system can be employed using two pulleys or wheels 60 as depicted in FIG. 6. The pulleys 60 may be motor driven and mounted in a housing. In one embodiment, the pulleys 60 are of different sizes. Rotation of the different sized pulleys 60 results in different rates of retraction of the pull wires and thus different rates of articulation. Alternatively, the pulleys may be of the same size but engage at different times to act on the pull ring 28 or pull ring 30 at different times.

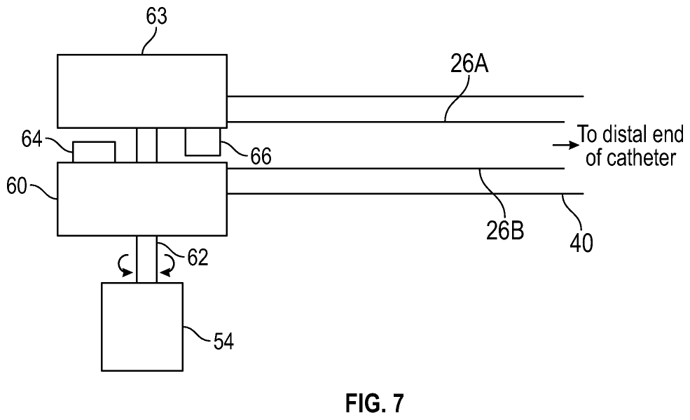

An alternative embodiment can be seen in FIG. 7 using a single motor 54 coupled to pulley 60 having via a shaft 62. The pulley 62 includes a cam 64. A second pulley 63 is not driven by the motor 54 but may also ride on the shaft 62. After some amount of rotation of the pulley 62, the cam 64 acts on a similar cam 66 on a second pulley 63. The second pulley 63 is only driven by the interaction of the cam 64 acting on cam 66. The result is that the first pulley 60 begins rotating before the cam 64 can act on the cam 66 to begin rotation of the second pulley 63. This delay in retraction of the pull wire 26A as compared to the immediate retraction of pull wire 26B achieves a similar articulation as the embodiments described in connection with FIG. 5.

The catheter 20, 40 may be utilized in as part of a system for intra-body navigation of a luminal network (e.g., the lungs of a patient). In accordance with the disclosure, a 3D volume of a patient's lungs or another suitable portion of the anatomy, may be generated from previously acquired scans, such as CT scans. These scans may be used to generate a 3D model of the anatomy. The 3D model and related scan data are used to identify targets, e.g., potential lesions for biopsy or treatment, and to generate a pathway plan through the anatomy to reach the targets.

Once the pathway plan is generated and accepted by a clinician, that pathway plan may be utilized by a navigation system to drive a catheter 20, 40 along the pathway plan through the anatomy to reach the desired target. The driving of the catheter 20, 40 along the pathway plan may be manual or it may be robotic, or a combination of both. Manual systems include the ILLUMISITE navigation system sold by Medtronic PLC, robotic systems include the ION system sold by Intuitive Surgical Inc. and the MONARCH system sold by Auris Health, Inc. In a single procedure planning, registration of the pathway plan to the patient, and navigation are performed to enable a medical device, e.g., a catheter 20, 40 to be navigated along the planned path to reach a target, e.g., a lesion, so that a biopsy or treatment of the target can be completed.

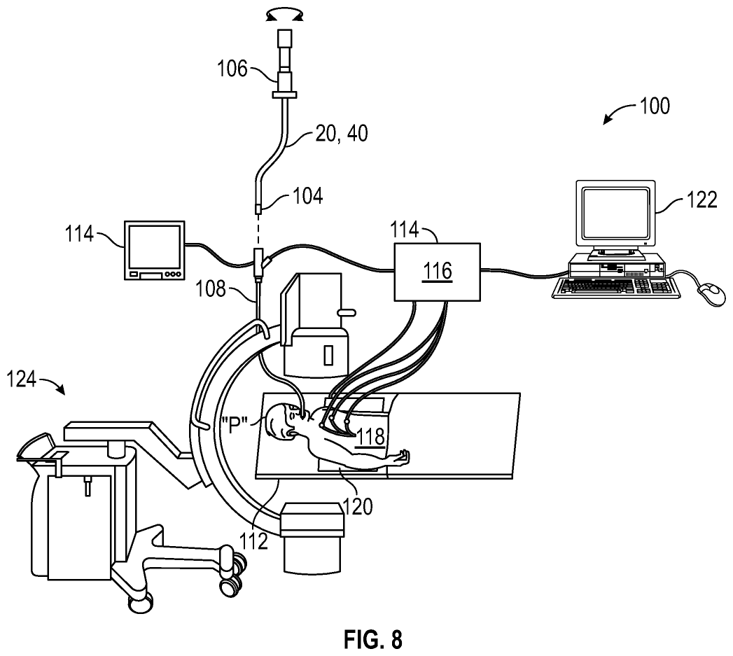

FIG. 8 is a perspective view of an exemplary system for facilitating navigation of a medical device, e.g., a catheter 20, 40 to a soft-tissue target via airways of the lungs. System 100 may be further configured to construct fluoroscopic based three-dimensional volumetric data of the target area from 2D fluoroscopic images to confirm navigation to a desired location. System 100 may be further configured to facilitate approach of a medical device to the target area by using Electromagnetic Navigation (EMN) and for determining the location of a medical device with respect to the target. One such EMN system is the ILLUMISITE system, though other systems for intraluminal navigation are considered within the scope of the disclosure, as noted above.

One aspect of the system 100 is a software component for reviewing of computed tomography (CT) image scan data that has been acquired separately from system 100. The review of the CT image data allows a user to identify one or more targets, plan a pathway to an identified target (planning phase), navigate a catheter 20, 40 to the target (navigation phase) using a user interface on computing device 122, and confirming placement of a sensor 104 housed in the catheter 20, 40 relative to the target. The target may be tissue of interest identified by review of the CT image data during the planning phase. Following navigation, a medical device, such as a biopsy tool or other tool, may be inserted into catheter 20, 40 to obtain a tissue sample from the tissue located at, or proximate to, the target.

As shown in FIG. 8, catheter 20, 40 is part of a catheter guide assembly 106. In practice, catheter 20, 40 may be inserted into a bronchoscope 108 for access to a luminal network of the patient P. Specifically, catheter 20, 40 of catheter guide assembly 106 may be inserted into a working channel of bronchoscope 108 for navigation through a patient's luminal network. Alternatively, the catheter guide assembly 106, may be navigated through the patient's luminal network without the use of a bronchoscope 108 without departing from the scope of the disclosure. A sensor 104 is located on the distal portion of the catheter 20, 40. The position and orientation of sensor 104 relative to a reference coordinate system, and thus the distal portion of catheter 20, 40, within an electromagnetic field can be derived.

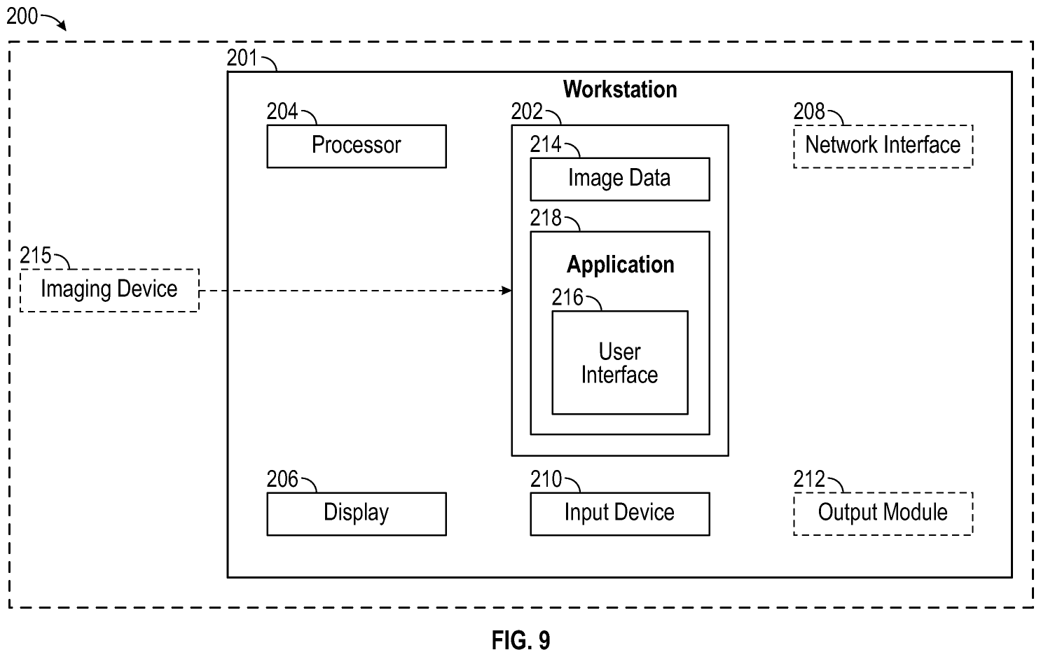

System 100 generally includes an operating table 112 configured to support a patient P, and monitoring equipment 114 coupled to bronchoscope 108 or the catheter guide assembly 106 (e.g., a video display, for displaying the video images received from the video imaging system of bronchoscope 108); a locating or tracking system 114 including a locating module 116, a plurality of reference sensors 118 and a transmitter mat 120 including a plurality of incorporated markers; and a computing device 122 including software and/or hardware used to facilitate identification of a target, pathway planning to the target, navigation of a medical device to the target, and/or confirmation and/or determination of placement of catheter 20, 40, or a suitable device therethrough, relative to the target. Computing device 122 may be similar to workstation 401 of FIG. 9.

A fluoroscopic imaging device 124 capable of acquiring fluoroscopic or x-ray images or video of the patient P is also included in this particular aspect of system 100. The images, sequence of images, or video captured by fluoroscopic imaging device 124 may be stored within fluoroscopic imaging device 124 or transmitted to computing device 122 for storage, processing, and display. Additionally, fluoroscopic imaging device 124 may move relative to the patient P so that images may be acquired from different angles or perspectives relative to patient P to create a sequence of fluoroscopic images, such as a fluoroscopic video. The pose of fluoroscopic imaging device 124 relative to patient P and while capturing the images may be estimated via markers incorporated with the transmitter mat 120. The markers are positioned under patient P, between patient P and operating table 112 and between patient P and a radiation source or a sensing unit of fluoroscopic imaging device 124. The markers incorporated with the transmitter mat 120 may be two separate elements which may be coupled in a fixed manner or alternatively may be manufactured as a single unit. Fluoroscopic imaging device 124 may include a single imaging device or more than one imaging device.

Computing device 122 may be any suitable computing device including a processor and storage medium, wherein the processor is capable of executing instructions stored on the storage medium. Computing device 122 may further include a database configured to store patient data, CT data sets including CT images, fluoroscopic data sets including fluoroscopic images and video, fluoroscopic 3D reconstruction, navigation plans, and any other such data. Although not explicitly illustrated, computing device 122 may include inputs, or may otherwise be configured to receive, CT data sets, fluoroscopic images/video and other data described herein. Additionally, computing device 122 includes a display configured to display graphical user interfaces. Computing device 122 may be connected to one or more networks through which one or more databases may be accessed.

With respect to the planning phase, computing device 122 utilizes previously acquired CT image data for generating and viewing a three-dimensional model or rendering of patient P's airways, enables the identification of a target on the three-dimensional model (automatically, semi-automatically, or manually), and allows for determining a pathway through patient P's airways to tissue located at and around the target. More specifically, CT images acquired from previous CT scans are processed and assembled into a three-dimensional CT volume, which is then utilized to generate a three-dimensional model of patient P's airways. The three-dimensional model may be displayed on a display associated with computing device 122, or in any other suitable fashion. Using computing device 122, various views of the three-dimensional model or enhanced two-dimensional images generated from the three-dimensional model are presented. The enhanced two-dimensional images may possess some three-dimensional capabilities because they are generated from three-dimensional data. The three-dimensional model may be manipulated to facilitate identification of target on the three-dimensional model or two-dimensional images, and selection of a suitable pathway through patient P's airways to access tissue located at the target can be made. Once selected, the pathway plan, three-dimensional model, and images derived therefrom, can be saved and exported to a navigation system for use during the navigation phase(s). The ILLUMISITE software suite currently sold by Medtronic PLC includes one such planning software.

With respect to the navigation phase, a six degrees-of-freedom electromagnetic locating or tracking system 114, or other suitable system for determining position and orientation of a distal portion of the catheter 20, 40, is utilized for performing registration of the images and the pathway for navigation. Tracking system 114 includes the tracking module 116, a plurality of reference sensors 118, and the transmitter mat 120 (including the markers). Tracking system 114 is configured for use with a catheter 20, 40 and particularly sensor 104.

Transmitter mat 120 is positioned beneath patient P. Transmitter mat 120 generates an electromagnetic field around at least a portion of the patient P within which the position of a plurality of reference sensors 118 and the sensor 104 can be determined with use of a tracking module 116. One or more of reference sensors 118 are attached to the chest of the patient P. Registration is generally performed to coordinate locations of the three-dimensional model and two-dimensional images from the planning phase, with the patient P's airways as observed through the bronchoscope 108, and allow for the navigation phase to be undertaken with knowledge of the location of the sensor 104.

Registration of the patient P's location on the transmitter mat 120 may be performed by moving sensor 104 through the airways of the patient P. More specifically, data pertaining to locations of sensor 104, while catheter 20, 40 is moving through the airways, is recorded using transmitter mat 120, reference sensors 118, and tracking system 114. A shape resulting from this location data is compared to an interior geometry of passages of the three-dimensional model generated in the planning phase, and a location correlation between the shape and the three-dimensional model based on the comparison is determined, e.g., utilizing the software on computing device 122. In addition, the software identifies non-tissue space (e.g., air filled cavities) in the three-dimensional model. The software aligns, or registers, an image representing a location of sensor 104 with the three-dimensional model and/or two-dimensional images generated from the three-dimension model, which are based on the recorded location data and an assumption that sensor 104 remains located in non-tissue space in patient P's airways. Alternatively, a manual registration technique may be employed by navigating the bronchoscope 108 with the sensor 104 to pre-specified locations in the lungs of the patient P, and manually correlating the images from the bronchoscope to the model data of the three-dimensional model.

As described herein above, the catheter 20, 40 may include one or more pull-wires 26 which can be used to manipulate the distal portion of the catheter 20, 40. The pull wires 26 may be manually operated, power assisted, and robotically drive. Though certain pull-wire systems are described here in detail, the disclosure is not so limited, the same principals of extension and retraction of pull wires may be employed by manual manipulation means to change the shape of the distal portion of the catheter without departing from the scope of the disclosure.

Though described herein with respect to EMN systems using EM sensors, the instant disclosure is not so limited and may be used in conjunction with shape sensors, flexible sensors, ultrasonic sensors, or with other types of sensors. Additionally, the methods described herein may be used in conjunction with robotic systems such that robotic actuators drive the catheter 102 or bronchoscope 108 proximate the target as described in greater detail below.

During navigation, upon arriving at a bifurcation, the catheter 20/40 is shaped to achieve a desired curve in the shape of the catheter 20/40 to enable rotation of the catheter 102 such that the distal end of the catheter 20/40 is aligned with a particular branch through which navigation is intended to proceed. Advancement of the catheter 20/40 then ensures with the curve so aligned ensures that navigation will continue in the desired branch. The curvature of the catheter 20/40 can be adjusted based on the need for curvature to reach a particular airway (e.g., when navigating the upper lobes of the lungs).

Reference is now made to FIG. 9, which is a schematic diagram of a system 200 configured for use with the methods of the disclosure including the method of FIG. 4. System 200 may include a workstation 201, and optionally a fluoroscopic imaging device or fluoroscope 215. In some embodiments, workstation 201 may be coupled with fluoroscope 215, directly or indirectly, e.g., by wireless communication. Workstation 201 may include a memory 202, a processor 204, a display 206 and an input device 210. Processor or hardware processor 204 may include one or more hardware processors. Workstation 201 may optionally include an output module 212 and a network interface 208. Memory 202 may store an application 218 and image data 214. Application 218 may include instructions executable by processor 204 for executing the methods of the disclosure.

Application 218 may further include a user interface 216. Image data 214 may include the CT scans, the generated fluoroscopic 3D reconstructions of the target area and/or any other fluoroscopic image data and/or the generated one or more slices of the 3D reconstruction. Processor 204 may be coupled with memory 202, display 206, input device 210, output module 212, network interface 208 and fluoroscope 215. Workstation 201 may be a stationary computing device, such as a personal computer, or a portable computing device such as a tablet computer. Workstation 201 may embed a plurality of computer devices.

Memory 202 may include any non-transitory computer-readable storage media for storing data and/or software including instructions that are executable by processor 204 and which control the operation of workstation 201 and, in some embodiments, may also control the operation of fluoroscope 215. Fluoroscope 215 may be used to capture a sequence of fluoroscopic images based on which the fluoroscopic 3D reconstruction is generated and to capture a live 2D fluoroscopic view according to this disclosure. In an embodiment, memory 202 may include one or more storage devices such as solid-state storage devices, e.g., flash memory chips. Alternatively, or in addition to the one or more solid-state storage devices, memory 202 may include one or more mass storage devices connected to the processor 204 through a mass storage controller (not shown) and a communications bus (not shown).

Although the description of computer-readable media contained herein refers to solid-state storage, it should be appreciated by those skilled in the art that computer-readable storage media can be any available media that can be accessed by the processor 204. That is, computer readable storage media may include non-transitory, volatile and non-volatile, removable and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by workstation 1001.

Application 218 may, when executed by processor 204, cause display 206 to present user interface 216. User interface 216 may be configured to present to the user a single screen including a three-dimensional (3D) view of a 3D model of a target from the perspective of a tip of a medical device, a live two-dimensional (2D) fluoroscopic view showing the medical device, and a target mark, which corresponds to the 3D model of the target, overlaid on the live 2D fluoroscopic view. User interface 216 may be further configured to display the target mark in different colors depending on whether the medical device tip is aligned with the target in three dimensions.

Network interface 208 may be configured to connect to a network such as a local area network (LAN) consisting of a wired network and/or a wireless network, a wide area network (WAN), a wireless mobile network, a Bluetooth network, and/or the Internet. Network interface 208 may be used to connect between workstation 201 and fluoroscope 215. Network interface 208 may be also used to receive image data 214. Input device 210 may be any device by which a user may interact with workstation 201, such as, for example, a mouse, keyboard, foot pedal, touch screen, and/or voice interface. Output module 212 may include any connectivity port or bus, such as, for example, parallel ports, serial ports, universal serial busses (USB), or any other similar connectivity port known to those skilled in the art. From the foregoing and with reference to the various figures, those skilled in the art will appreciate that certain modifications can be made to the disclosure without departing from the scope of the disclosure.

While detailed embodiments are disclosed herein, the disclosed embodiments are merely examples of the disclosure, which may be embodied in various forms and aspects. For example, embodiments of an electromagnetic navigation system, which incorporates the target overlay systems and methods, are disclosed herein; however, the target overlay systems and methods may be applied to other navigation or tracking systems or methods known to those skilled in the art. Therefore, specific structural and functional details disclosed herein are not to be interpreted as limiting, but merely as a basis for the claims and as a representative basis for teaching one skilled in the art to variously employ the disclosure in virtually any appropriately detailed structure.

We claim:

1. An intraluminal navigation catheter comprising:
a catheter tube;
a first pull ring secured to the catheter tube and located proximate a distal end of the catheter tube;
a second pull ring secured to the catheter tube, the second pull ring located proximal of the first pull ring and including a lumen formed therein,
a pull wire permanently secured to the first pull ring;
a stopper affixed to the pull wire between the first pull ring and the second pull ring, the stopper sized to prevent travel through the lumen in the second pull wire; and
an actuator operably connected to the pull wire, wherein the actuator is configured to retract the pull wire to initially induce a curve in a distal portion of the catheter and upon further retraction impinge the stopper against the second pull ring to induce a curve in the catheter in a portion proximal of the first curve.

2. The intraluminal navigation catheter of claim 1, wherein the actuator is motor driven.

3. The intraluminal navigation catheter of claim 2, wherein the motor is robotically initialized.

4. The intraluminal navigation catheter of claim 2, wherein the motor is manually initialized.

5. The intraluminal navigation catheter of claim 1, wherein the stopper is cross shaped.

6. The intraluminal navigation catheter of claim 1, wherein pull wire is secured to a shuttle operably connected to a leadscrew, the leadscrew driven by a motor to advance and retract the pull wire.

7. The intraluminal navigation catheter of claim 1, wherein the pull wire is secured to a pulley, wherein rotation of the pulley by a motor retracts or advances the pull wire.

8. An intraluminal navigation catheter comprising:
a catheter tube;
a first pull ring secured to the catheter tube and located proximate a distal end of the catheter tube;
a second pull ring secured to the catheter tube, the second pull ring located proximal of the first pull ring and including a lumen formed therein,
a first pull wire permanently secured to the first pull ring;
a second pull wire permanently secured to the second pull ring;
an actuator operably connected to the first pull wire and the second pull wire, wherein the actuator is configured to retract the first pull wire to induce a first curve in a distal portion of the catheter and operably connected to the second pull wire to retract the second pull wire to induce a curve in a portion of the catheter proximal of the first curve.

9. The intraluminal navigation catheter of claim 8 further comprising a shuttle fixedly connected to the first pull wire and slidingly connected to the second pull wire.

10. The intraluminal navigation catheter of claim 9, further comprising a stopper formed on the second pull wire.

11. The intraluminal navigation catheter of claim 10, wherein the stopper is formed on a proximal end of the second pull wire, wherein the shuttle is operably connected to a leadscrew driven by a motor, wherein retraction of the shuttle actuates the first pull wire to induce the curve at the distal end of the catheter, and further retraction impinges the stopper against the shuttle to induce a curve in the catheter in a portion proximal of the first curve.

12. The intraluminal navigation catheter of claim 8 further comprising two pulleys, one operably connected to the first pull wire and one operably connected to the second pull wire, wherein retraction of the second pull wire is commenced after retraction of the first pull wire.

13. The intraluminal navigation catheter of claim 8 further comprising two pulleys, a first pulley operably connected to the first pull wire and a second pulley operably connected to the second pull wire, wherein the second pulley is smaller than the first pulley.

14. The intraluminal navigation catheter of claim 8 further comprising two pulleys, a first pulley operably connected to the first pull wire and a second pulley operably connected to the second pull wire, the first pulley including a first cam and the second pulley including a second cam, wherein rotation of the first pulley causes the first cam to act on the second cam to achieve rotation of the second pulley.

15. An intraluminal navigation system comprising:
a catheter including
a catheter tube;
a sensor formed in a distal portion of the catheter tube;
a first pull ring secured to the catheter tube and located proximate a distal end of the catheter tube;
a second pull ring secured to the catheter tube, the second pull ring located proximal of the first pull ring and including a lumen formed therein,
a pull wire permanently secured to the first pull ring;
a stopper affixed to the pull wire between the first pull ring and the second pull ring, the stopper sized to prevent travel through the lumen in the second pull wire;
a motor drive actuator operably connected to the pull wire, wherein the motor driven actuator is configured to retract the pull wire to induce a curve in a distal portion of the catheter and upon further retraction impinge the stopper against the second pull ring to induce a curve in the catheter in a portion proximal of the first curve
a tracking system configured to detect the position of the sensor; and
a computing device configured to depict the detected position of the sensor in a user interface on a display.

16. The intraluminal navigation system of claim 15 wherein the user interface displays one or more of a three-dimensional model, fluoroscopic images, computer tomographic images of a luminal network and the position of the sensor in the luminal network.

17. The intraluminal navigation system of claim 15, wherein the sensor is an electromagnetic sensor.

18. The intraluminal navigation system of claim 15, wherein the sensor is a shape sensor.

19. The intraluminal navigation system of claim 15, wherein the motor driven actuator is robotically driven.

20. The intraluminal navigation of claim 15, further comprising a shuttle operably connected to the pull wire.
